(19) United States
(12) Patent Application Publication
(10) Pub. No.: US 2024/0173362 A1
(43) Pub. Date: May 30, 2024

(54) ANTIBIOTIC-RESISTANT PROBIOTICS AND USES THEREOF

(71) Applicant: SUPERBREWED FOOD, INC., New Castle, DE (US)

(72) Inventors: Alon KARPOL, Tel Mond (IL); Racheli Tamir, Sugar Land, TX (US); Aharon M. EYAL, Jerusalem (IL); Bryan P. TRACY, Wilmington, DE (US)

(21) Appl. No.: 18/280,963

(22) PCT Filed: Mar. 9, 2022
(86) PCT No.: PCT/IB2022/052112

§ 371 Date: Sep. 8, 2023

Related U.S. Application Data
(60) Provisional application No. 63/159,082, filed on Mar. 10, 2021.

(51) Int. Cl. A61K 35/742; A61K 35/745; A61K 35/747; A61K 36/064; A61P 1/12; A61K 35/00

(52) U.S. Cl. CPC A61K 35/742; A61K 35/745; A61K 35/747; A61K 36/064; A61P 1/12; A61K 2035/115

(57) ABSTRACT

Provided are compositions and kits comprising at least one probiotic microorganism which is resistant to an antibiotic drug and uses thereof in the treatment of conditions caused by administration of the same antibiotic, wherein the probiotic microorganism and the antibiotic drug may be administered separately or provided together in a single dosage form.

### CROSS-REFERENCE TO RELATED APPLICATION

The present application gains priority from U.S. Provisional Application No. 63/159,082 filed 10 Mar. 2021 which is incorporated by reference as if fully set-forth herein.

### FIELD OF THE INVENTION

The present invention, in at least some embodiments, relates to antibiotic-resistant probiotic microorganisms and to uses thereof in treating or preventing a condition caused by administration of an antibiotic drug.

### BACKGROUND OF THE INVENTION

Antibiotics are commonly used to treat or prevent a wide range of bacterial infections.

Use of antibiotics is known to cause various side-effects. The most common side effects are those which affect the digestive system, such as nausea, indigestion, vomiting, diarrhea, bloating, loss of appetite and stomach cramping or pain.

### SUMMARY OF THE INVENTION

According to an aspect of some embodiments of the present invention, there is provided a method for treating or preventing a condition caused by administration of an antibiotic drug in a subject in need thereof, the method comprising administering to said subject an effective dose of at least one probiotic microorganism which is resistant to said antibiotic drug.

According to a further aspect of some embodiments of the present invention, there is provided a kit comprising an effective dose of an antibiotic drug and an effective dose of at least one probiotic microorganism which is resistant to said antibiotic drug.

According to a further aspect of some embodiments of the present invention, there is provided a composition comprising an effective dose of an antibiotic and an effective dose of at least one probiotic microorganism which is resistant to said antibiotic drug.

### DETAILED DESCRIPTION OF THE INVENTION

The present invention relates to compositions and kits comprising antibiotic-resistant probiotic microorganisms and uses thereof in treatment of conditions caused by use of antibiotics.

The present inventors have surprisingly found that at least one undesirable side effect in a subject caused by administration of an antibiotic can be partially or fully treated or prevented by administering to the subject a probiotic microorganism which is resistant to the antibiotic causing the at least one undesirable side effect.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used herein, the term “treating” includes preventing, curing, ameliorating, mitigating, and reducing the instances or severity of a condition or a symptom thereof.

As used herein, the term “administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, intramuscular, intraosseous, intrathecal, and transdermal, or combinations thereof. “Administering” can also include providing a different compound that when ingested or delivered as above will necessarily transform into the compound that is desired to be administered, this type of “different compound” is often being referred to as a “Prodrug”. “Administering” can also include prescribing or filling a prescription for a dosage form comprising a particular compound. “Administering” can also include providing directions to carry out a method involving a particular compound or a dosage form comprising the compound or compounds.

As used herein, the term “effective dose” means the amount of an active substance that, when administered to a subject for treating a disease, disorder, or other undesirable medical condition, is sufficient to have a beneficial effect with respect to that disease, disorder, or condition. The effective dose will vary depending on the identity and formulation form of the active substance, the disease or condition and its severity, and the age, weight, and other relevant characteristics of the patient to be treated. Determining the effective amount of a given active substance is within the ordinary skill of the art and typically requires no more than routine experimentation.

As used herein, the term “probiotic microorganism” refers to a live microorganism which provides health benefits to an animal when consumed, generally by restoring the balance of gut flora.

As used herein, the term “resistant” with regard to an antibiotic drug refers to a microorganism which has the ability to survive and replicate in the presence of at least a minimum inhibitory concentration (MIC) of the antibiotic drug according to the microbiological cut-off (mg/L) values determined by the European Food Safety Authority (EFSA), for example such as those set out in Table 1 below.

TABLE 1
Table [symbol] : Microbiological cut-off values (mg/L)

| | [symbol] | [symbol] | [symbol] | [symbol] | [symbol] | [symbol] | [symbol] | [symbol] | [symbol] | [symbol] | [symbol] | [symbol] | [symbol] |
|---|---|---|---|---|---|---|---|---|---|---|---|---|---|
| [symbol] | 2 | 2 | 16 | [symbol] | [symbol] | 1 | 4 | 4 | 4 | [symbol] | [symbol] | [symbol] | [symbol] |
| [symbol] | 1 | 2 | [symbol] | 64 | [symbol] | 1 | 4 | 4 | 4 | [symbol] | [symbol] | [symbol] | [symbol] |
| [symbol] | 2 | [symbol] | [symbol] | 64 | [symbol] | 1 | 4 | [symbol] | 4 | [symbol] | [symbol] | [symbol] | [symbol] |
| [symbol] | 2 | [symbol] | 8 | 64 | [symbol] | 1 | 4 | 27 | 4 | [symbol] | [symbol] | [symbol] | [symbol] |
| [symbol] | 4 | [symbol] | 16 | 64 | [symbol] | 1 | 4 | 8 | 4 | [symbol] | [symbol] | [symbol] | [symbol] |
| [symbol] | 2 | [symbol] | 16 | 64 | [symbol] | 1 | 4 | 32 | 8 | [symbol] | [symbol] | [symbol] | [symbol] |
| [symbol] | 4 | [symbol] | [symbol] | 64 | [symbol] | 1 | 4 | [symbol] | 4 | [symbol] | [symbol] | [symbol] | [symbol] |
| [symbol] | 4 | [symbol] | 32 | 64 | [symbol] | 1 | 4 | 4 | 4 | [symbol] | [symbol] | [symbol] | [symbol] |
| [symbol] | 2 | [symbol] | [symbol] | [symbol] | [symbol] | 1 | 1 | 8 | 4 | [symbol] | [symbol] | [symbol] | [symbol] |
| [symbol] | 4 | [symbol] | 16 | 64 | [symbol] | 1 | 1 | 8 | 4 | [symbol] | [symbol] | [symbol] | [symbol] |
| [symbol] | 2 | [symbol] | [symbol] | 16 | [symbol] | 1 | 1 | 8 | [symbol] | [symbol] | [symbol] | [symbol] | [symbol] |
| [symbol] | 2 | 4 | 32 | [symbol] | [symbol] | 1 | 1 | 4 | [symbol] | [symbol] | [symbol] | [symbol] | [symbol] |
| [symbol] | 2 | 4 | [symbol] | [symbol] | [symbol] | 2 | 2 | 4 | 4 | [symbol] | [symbol] | [symbol] | [symbol] |
| [symbol] | [symbol] | 4 | 4 | 8 | 8 | 4 | 4 | [symbol] | [symbol] | [symbol] | [symbol] | [symbol] | [symbol] |
| [symbol] | 2 | 4 | 64 | 64 | 64 | 0.5 | 0.25 | [symbol] | [symbol] | [symbol] | [symbol] | [symbol] | [symbol] |
| [symbol] | 2 | 4 | 32 | [symbol] | [symbol] | 4 | 4 | 4 | [symbol] | 4 | [symbol] | [symbol] | [symbol] |
| [symbol] | 1 | 4 | 4 | 16 | 8 | 1 | 4 | 2 | 4 | [symbol] | [symbol] | [symbol] | [symbol] |
| [symbol] | 8 | [symbol] | 2 | 8 | [symbol] | [symbol] | [symbol] | [symbol] | [symbol] | [symbol] | [symbol] | 2 | 8 |

[symbol]

[symbol]

[symbol]

[symbol]

[symbol]

[symbol]

indicates data missing or illegible when filed

Antibiotic resistance in a microorganism may be determined using any method known in the art, including but not limited to Antibiotic Susceptibility Testing.

As used herein, the term “acetogen” refers to an obligately anaerobic bacterium that uses the reductive acetyl-CoA or Wood-Ljungdahl pathway as its main mechanism for energy conservation and for synthesis of acetyl-CoA and cell carbon from CO₂.

As used herein, the term “spore former” refers to a microorganism capable of forming spores, which are highly resistant, dormant structures having no metabolic activity, formed in some microorganisms in response to environmental conditions (such as depletion of nutrients) in order to help in survival of the microorganism.

As used herein, the term “vegetative cell” refers to a bacterial cell that is actively growing rather than forming spores.

As used herein, the term “transferable genetic element” (also known as “mobile genetic elements”) are a type of genetic material that can move around within a genome, or that can be transferred from one species or replicon to another. Examples of transferable genetic elements include plasmids, transposons, viruses, introns and integrons.

As used herein, the term “prebiotic” refers to a compound that induces the growth or activity of a probiotic.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, there is provided a method for treating or preventing a condition caused by administration of an antibiotic drug in a subject in need thereof, the method comprising administering to said subject an effective dose of at least one probiotic microorganism which is resistant to said antibiotic drug.

According to some embodiments, said microorganism is of the class clostridia.

According to some embodiments, said microorganism is of an order selected from the group consisting of Clostridiales, Halanaerobiales, Natranaerobiales, Thermoanaerobacteriales and combinations thereof.

According to some embodiments, said at least one probiotic microorganism comprises two or more different types of microorganisms (such as two, three, four, five or more different types of microorganisms).

According to some embodiments, each type of microorganism is selected from a different order. According to some embodiments, each type of microorganism is selected from the same order.

According to some embodiments, each type of microorganism is selected from a different genus. According to some embodiments, each type of microorganism is selected from the same genus.

According to some embodiments, each type of microorganism is selected from a different group. According to some embodiments, each type of microorganism is selected from the same group.

According to some embodiments, said microorganism is an acetogen.

According to some embodiments, said microorganism is of a genus selected from the group consisting of Acetitomaculum; Acetoanaerobium; Acetobacterium; Acetohalobium; Acetoneme; Bacillus; Blautia; Bryantella; Butyribacterium; Caloramator; Clostridium; Desulfovibrio; Enterococcus; Eubacterium; Gottschalkia; Holophage; Methylobacterium; Micrococcus; Moorella; Mycobacterium; Natronielle; Natronincola; Oxobacter; Peptoniphilus; Proteus; Reticulitermes; Rhizobium; Ruminococcus; Saccharomyces; Sinorhizobium; Sphingomonas; Sporomusa; Syntrophococcus; Thermoacetogenium; Tindallia; Treponema; Veillonella and combinations thereof.

According to some embodiments, said microorganism is a spore former.

According to some embodiments, said microorganism is a vegetative cell.

According to some embodiments, said microorganism comprises both spores and vegetative cells.

According to some embodiments, said resistance to said antibiotic drug in said microorganism is naturally occurring.

According to some embodiments, said resistance to said antibiotic drug in said organism is not on a transferable genetic element such as a plasmid, transposon, virus, intron or integron.

According to some embodiments, said microorganism is obtained artificially by controlled selection or enrichment.

According to some embodiments, said microorganism is genetically modified.

According to some embodiments, said at least one antibiotic drug is selected from the group consisting of Aminoglycosides, Carbapenems, Cephalosporins, Fluoroquinolones, Glycopeptides, lipoglycopeptides, Macrolides, Monobactams, Oxazolidinones, Penicillin, Polypeptides, Rifamycin, Sulfonamides, Streptogramins, Tetracyclines, Ampicillin, clindamycin and combinations thereof.

According to some embodiments, said antibiotic drug is selected from the group consisting of Penicillin, Tetracyclines, Ampicillin, clindamycin and combinations thereof.

According to some embodiments, said microorganism is provided in a dosage form selected from the group consisting of a capsule, a pill, an emulsion and an enema. According to some embodiments, said antibiotic is provided in a dosage form selected from the group consisting of a capsule, a pill, an emulsion and an enema.

According to some embodiments, said microorganism is provided in the form of a capsule having a weight of from about 0.1 to about 0.1 gram, wherein said capsule comprises at least 104 colony forming units (CFU) of said microorganism/gram.

According to some embodiments, said microorganism is administered in a single dosage form together with said antibiotic.

According to some embodiments, said microorganism is administered in a separate dosage form to said antibiotic.

According to some embodiments, wherein said microorganism is administered in a separate dosage form to said antibiotic, said microorganism and said antibiotic are administered independently, sequentially, substantially simultaneously, or concomitantly. In some embodiments, said microorganism is administered prior to administration of said antibiotic. In some embodiments, said microorganism is administered after administration of said antibiotic.

According to some embodiments, wherein said microorganism and said antibiotic are administered in separate dosage forms, the separate dosage forms may be separate dosage forms of the same type e.g. each of said microorganism and said antibiotic are administered separately as a capsule. Alternatively, the separate dosage forms may be separate dosage forms of different types e.g. said microorganism may be administered as an emulsion and said antibiotic may be administered as a pill.

According to some embodiments, the method further comprises administering a prebiotic, such as a fibre selected from the group consisting of fructooligosaccharides (FOS), inulins, galactooligosaccharides (GOS), resistant starch, pectin, beta-glucans, xylooligosaccharides, mannan oligosaccharide and combinations thereof.

According to some embodiments, said prebiotic is administered together with said probiotic microorganism and/or said antibiotic in a single dosage form. According to some embodiments, said prebiotic is administered separately from said probiotic microorganism and/or said antibiotic. According to some embodiments, wherein said prebiotic is administered in a separate dosage form to said antibiotic and/or said microorganism, said microorganism, said antibiotic and said prebiotic are administered independently, sequentially, substantially simultaneously, or concomitantly. According to some such embodiments, said microorganism, said antibiotic and said prebiotic are administered independently or sequentially in any order.

According to some embodiments, the method further comprises administering a salt or an ester of butyric acid, such as tributyrin or a magnesium or calcium salt of butyric acid. According to an embodiment, said butyric acid salt is administered in a single dose of from about 100 to about 800 mg, such as about 100, about 150, about 200, about 250, about 300, about 350, about 400, about 450, about 500, about 550, about 600, about 650, about 700, about 750 or about 800 mg. According to some embodiments, said tributyrin is administered in a single dose of from about 70 mg to about 600 mg, such as about 70, about 100, about 150, about 200, about 250, about 300, about 350, about 400, about 450, about 500, about 550, or about 600 mg.

According to some embodiments, said salt or ester of butyric acid is administered together with said probiotic microorganism and/or said antibiotic and/or said prebiotic in a single dosage form. According to some embodiments, said salt or ester of butyric acid is administered separately from said probiotic microorganism and/or said antibiotic and/or said prebiotic. According to some embodiments, wherein said salt or ester of butyric acid is administered in a separate dosage form to said antibiotic and/or said microorganism and/or said prebiotic, said microorganism, said antibiotic, said prebiotic and said salt or ester of butyric acid are administered independently, sequentially, substantially simultaneously, or concomitantly. According to some such embodiments, said microorganism, said antibiotic, said prebiotic and said salt or ester of butyric acid are administered independently or sequentially in any order.

According to some embodiments, said method further comprises administering a microorganism of the class clostridia and an additional microorganism selected from the group consisting of Lactobacillus, Pediococcus, Streptococcus, Bifidobacterium, Enterococcus, Bacillus, Saccharomyces, Propionibacterium and combinations thereof. In some embodiments, said additional microorganism and said microorganism of the class clostridia are administered in a single dosage form. In some embodiments, said additional microorganism and said microorganism of the class clostridia are administered independently, sequentially, substantially simultaneously, or concomitantly in separate dosage forms.

According to some embodiments, said administering is carried out multiple times per day, such as twice, three times, four times or six times per day.

According to some embodiments, said administering is carried out for a period of one days, two days, three days, four days, five days, six days, one week, two weeks, three weeks, or one month.

According to some embodiments, said administering of said microorganism is carried out for the same time period as that of administering of said antibiotic. According to some embodiments, said administering of said microorganism is carried out for a shorter time period than that of administering of said antibiotic. According to some embodiments, said administering of said microorganism is carried out for a longer time period as that of administering of said antibiotic. According to some embodiments, said administering of said microorganism commences prior to commencement of administering of said antibiotic. According to some embodiments, said administering of said microorganism commences subsequent to commencement of administering of said antibiotic.

According to some embodiments, said microorganism is administered prior to administration of said antibiotic to a subject having a known susceptibility to at least one side effect of said antibiotic.

According to some embodiments, said condition comprises Antibiotic Associated Diarrhea (AAD).

According to some embodiments wherein said conditions comprises AAD, said method further comprises administering an anti-diarrheal agent.

According to some embodiments, said anti-diarrheal agent is administered together with said probiotic microorganism and/or said antibiotic and/or said prebiotic and/or said salt or ester of butyric acid in a single dosage form. According to some embodiments, said anti-diarrheal agent is administered separately from said probiotic microorganism and/or said antibiotic and/or said prebiotic and/or said salt or ester of butyric acid.

According to some embodiments, wherein said anti-diarrheal agent is administered in a separate dosage form to said antibiotic and/or said microorganism and/or said prebiotic and/or said salt or ester of butyric acid, said microorganism, said antibiotic, said prebiotic, said salt or ester of butyric acid and said anti-diarrheal agent are administered independently, sequentially, substantially simultaneously, or concomitantly. According to some such embodiments, said microorganism, said antibiotic, said prebiotic, said salt or ester of butyric acid and said anti-diarrheal agent are administered independently or sequentially in any order.

According to some embodiments, said microorganism is encapsulated. According to some such embodiments, encapsulation is achieved by use of at least one selected from the group consisting of alginate carrageenan, gelatin, chitosan, whey proteins, cellulose acetate phthalate, locust bean gum, starch and combinations thereof.

According to some embodiments, the method further comprises administering a probiotic which is not resistant to said antibiotic drug, wherein said probiotic which is not resistant to said antibiotic drug is administered at least 24 hours before or after administration of said antibiotic drug.

According to an aspect of some embodiments of the present invention, there is provided a kit comprising an effective dose of an antibiotic drug and an effective dose of at least one probiotic microorganism which is resistant to said antibiotic drug.

According to an aspect of some embodiments of the present invention, there is provided a composition comprising an effective dose of an antibiotic and an effective dose of at least one probiotic microorganism which is resistant to said antibiotic drug.

According to an aspect of some embodiments of the present invention, there is provided a composition comprising an effective dose of an antibiotic drug for coadministration together with an effective dose of at least one probiotic microorganism which is resistant to said antibiotic drug.

According to some embodiments of the kit or composition, said microorganism is of the class clostridia.

According to some embodiments of the kit or composition, said microorganism is of an order selected from the group consisting of Clostridiales, Halanaerobiales, Natranaerobiales, Thermoanaerobacteriales.

According to some embodiments of the kit or composition, said at least one probiotic microorganism comprises two or more different types of microorganisms (such as two, three, four, five or more different types of microorganisms).

According to some embodiments of the kit or composition, each type of microorganism is selected from a different order. According to some embodiments of the kit or composition, each type of microorganism is selected from the same order.

According to some embodiments of the kit or composition, each type of microorganism is selected from a different genus. According to some embodiments of the kit or composition, each type of microorganism is selected from the same genus.

According to some embodiments of the kit or composition, each type of microorganism is selected from a different group. According to some embodiments of the kit or composition, each type of microorganism is selected from the same group.

According to some embodiments of the kit or composition, said microorganism is an acetogen.

According to some embodiments of the kit or composition, said microorganism is of a genus selected from the group consisting of Acetitomaculum; Acetoanaerobium; Acetobacterium; Acetohalobium; Acetoneme; Bacillus; Blautia; Bryantella; Butyribacterium; Caloramator; Clostridium; Desulfovibrio; Enterococcus; Eubacterium; Gottschalkia; Holophage; Methylobacterium; Micrococcus; Moorella; Mycobacterium; Natronielle; Natronincola; Oxobacter; Peptoniphilus; Proteus; Reticulitermes; Rhizobium; Ruminococcus; Saccharomyces; Sinorhizobium; Sphingomonas; Sporomusa; Syntrophococcus; Thermoacetogenium; Tindallia; Treponema; Veillonella and combinations thereof.

According to some embodiments of the kit or composition, said microorganism is a spore former.

According to some embodiments of the kit or composition, said microorganism is a vegetative cell.

According to some embodiments of the kit or composition, said microorganism comprises both spores and vegetative cells.

According to some embodiments of the kit or composition, said resistance to said antibiotic drug in said microorganism is naturally occurring.

According to some embodiments of the kit or composition, said resistance is not on a transferable genetic element such as a plasmid, transposon, virus, intron or integron.

According to some embodiments of the kit or composition, said microorganism is obtained artificially by controlled selection or enrichment.

According to some embodiments of the kit or composition, microorganism is genetically modified.

According to some embodiments of the kit or composition, said at least one antibiotic drug is selected from the group consisting of Aminoglycosides, Carbapenems, Cephalosporins, Fluoroquinolones, Glycopeptides, lipoglycopeptides, Macrolides, Monobactams, Oxazolidinones, Penicillin, Polypeptides, Rifamycin, Sulfonamides, Streptogramins, Tetracyclines, Ampicillin, clindamycin and combinations thereof.

According to some embodiments of the kit or composition, said antibiotic drug is selected from the group consisting of Penicillin, Tetracyclines, Ampicillin, clindamycin and combinations thereof.

According to some embodiments, the kit or composition further comprises at least one prebiotic. According to some embodiments of the kit, said prebiotic is provided in a separate dosage form from said microorganism and/or said antibiotic drug. According to some embodiments of the kit, said prebiotic is provided in a same dosage form as said microorganism and/or said antibiotic drug.

According to some embodiments, the kit or composition further comprises a salt or an ester of butyric acid, such as tributyrin or a magnesium or calcium salt of butyric acid. According to an embodiment, said composition or kit comprises from about 100 to about 800 mg, such as about 100, about 150, about 200, about 250, about 300, about 350, about 400, about 450, about 500, about 550, about 600, about 650, about 700, about 750 or about 800 mg of the salt of butyric acid per single dose. According to some embodiments, said composition or kit comprises from about 70 mg to about 600 mg tributyrin, such as about 70, about 100, about 150, about 200, about 250, about 300, about 350, about 400, about 450, about 500, about 550, or about 600 mg per single dose.

According to some embodiments of the kit, said salt or ester of butyric acid is provided in a separate dosage form from said microorganism and/or said antibiotic drug and/or said prebiotic. According to some embodiments of the kit, said salt or ester of butyric acid is provided in a same dosage form as said microorganism and/or said antibiotic drug and/or said prebiotic.

According to some embodiments, the kit or composition comprises a microorganism of the class clostridia and further comprises an additional probiotic microorganism selected from the group consisting of Lactobacillus, Pediococcus, Streptococcus, Bifidobacterium, Enterococcus, Bacillus, Saccharomyces, Propionibacterium and combinations thereof.

According to some embodiments, the kit or composition further comprises an anti-diarrheal agent. According to some embodiments of the kit, said anti-diarrheal agent is provided in a separate dosage form from said microorganism and/or said antibiotic drug and/or said prebiotic and/or said salt or ester of butyric acid. According to some embodiments of the kit, said prebiotic is provided in a same dosage form as said microorganism and/or said antibiotic drug and/or said prebiotic and/or said salt or ester of butyric acid.

According to some embodiments of the kit or composition, said microorganism is encapsulated. According to some such embodiments, encapsulation is achieved by use of at least one selected from the group consisting of alginate carrageenan, gelatin, chitosan, whey proteins, cellulose acetate phthalate, locust bean gum, starch and combinations thereof.

According to some embodiments, the kit or composition further comprises a microorganism which is not resistant to said antibiotic agent.

According to some embodiments, the composition is provided in a dosage form selected from the group consisting of a capsule, a pill, an emulsion and an enema. According to some embodiments of the kit, said microorganism and/or said antibiotic agent is provided in a dosage form selected from the group consisting of a capsule, a pill, an emulsion and an enema.

According to some embodiments of the kit or composition, said microorganism is provided in the form of a capsule having a weight of from about 0.1 to about 0.1 gram, wherein said capsule comprises at least 104 colony forming units (CFU) of said microorganism/gram.

According to some embodiments of the kit, each of said antibiotic agent and said microorganism are provided within a packaging in discrete dosage forms, wherein said discrete dosage forms are arranged in a specific sequence in said packaging.

According to an aspect of some embodiments of the present invention, there is provided the kit or composition as disclosed herein for use in the treatment of AAD.

According to an aspect of some embodiments of the present invention, there is provided the composition as disclosed herein for treating a condition caused by an antibiotic, by co-administration together with said antibiotic. According to an embodiment, co-administration may be carried out independently, sequentially, substantially simultaneously, or concomitantly in separate dosage forms or by administering in a single dosage form.

### Example

A clinical trial is conducted with a group of 200 subjects (100 males, 100 females) in the age range of 15-75 years. A placebo probiotic in the form of a pill containing excipient alone is administered to 50% of the subjects (control group), while a probiotic pill comprising, in addition to excipients, 109 colony forming units (CFU) of an acetogen of the class Clostridia having resistance to tetracycline is administered to the other 50% of subjects (test group).

24 hours after administration of the placebo or probiotic pill, subjects in both groups are treated with tetracycline antibiotic at a dose of 250 mg/day, and treatment is continued for an additional 7 days, using the same antibiotic dose.

During the treatment period, all subjects report some degree of the following side effects: bloating, diarrhea, nausea, indigestion, vomiting, loss of appetite, and stomach cramping or pain. Antibiotic-associated diarrhea (AAD) is reported for 20 subjects in the control group (i.e. 40% of subjects). In the test group, AAD is reported in 5 patients (i.e. 10%). Additionally, a reduction in bloating (50% reduction), stomach cramping and pain (40% reduction) is reported in the test group as compared to the control group.

1-47. (canceled)

48. A method for treating or preventing a condition caused by administration of an antibiotic drug in a subject in need thereof, the method comprising administering to said subject an effective dose of at least one probiotic microorganism of the class clostridia which is resistant to said antibiotic drug.

49. The method of claim 48, wherein said microorganism is of an order selected from the group consisting of Clostridiales, Halanaerobiales, Natranaerobiales, Thermoanaerobacteriales.

50. The method of claim 48, wherein said microorganism is an acetogen.

51. The method of claim 50, wherein said microorganism is of a genus selected from the group consisting of Acetitomaculum; Acetoanaerobium; Acetobacterium; Acetohalobium; Acetoneme; Bacillus; Blautia; Bryantella; Butyribacterium; Caloramator; Clostridium; Desulfovibrio; Enterococcus; Eubacterium; Gottschalkia; Holophage; Methylobacterium; Micrococcus; Moorella; Mycobacterium; Natronielle; Natronincola; Oxobacter; Peptoniphilus; Proteus; Reticulitermes; Rhizobium; Ruminococcus; Saccharomyces; Sinorhizobium; Sphingomonas; Sporomusa; Syntrophococcus; Thermoacetogenium; Tindallia; Treponema; Veillonella and combinations thereof.

52. The method of claim 48, wherein said microorganism is selected from the group consisting of a spore former, a vegetative cell and a combination thereof.

53. The method of claim 48, wherein said resistance to said antibiotic drug in said microorganism is naturally occurring.

54. The method of claim 53, wherein said resistance to said antibiotic drug in said organism is not on a transferable genetic element.

55. The method of claim 48, wherein said microorganism is obtained artificially by controlled selection or enrichment.

56. The method of claim 48, wherein said microorganism is genetically modified.

57. The method of claim 48, wherein said at least one antibiotic drug is selected from the group consisting of Aminoglycosides, Carbapenems, Cephalosporins, Fluoroquinolones, Glycopeptides, lipoglycopeptides, Macrolides, Monobactams, Oxazolidinones, Penicillin, Polypeptides, Rifamycin, Sulfonamides, Streptogramins, Tetracyclines, Ampicillin, Clindamycin and combinations thereof.

58. The method of claim 48, further comprising administering at least one selected from the group consisting of a prebiotic and a salt or an ester of butyric acid.

59. The method of claim 49, further comprising administering an additional probiotic microorganism selected from the group consisting of Lactobacillus, Pediococcus, Streptococcus, Bifidobacterium, Enterococcus, Bacillus, Saccharomyces, Propionibacterium and combinations thereof.

60. The method of claim 48, wherein said microorganism is provided in a dosage form selected from the group consisting of a capsule, a pill, an emulsion and an enema.

61. The method of claim 48, wherein said condition comprises Antibiotic Associated Diarrhea (AAD), optionally further comprising administering an anti-diarrheal agent.

62. The method of claim 48, wherein said microorganism is encapsulated.

63. The method of claim 48, further comprising administration of a probiotic which is not resistant to said antibiotic drug, wherein said probiotic which is not resistant to said antibiotic drug is administered at least 24 hours before or after administration of said antibiotic drug.

64. A kit comprising an effective dose of an antibiotic drug and an effective dose of at least one probiotic microorganism of the class clostridia which is resistant to said antibiotic drug.

65. The method of claim 48, wherein said antibiotic drug and said probiotic microorganism of the class clostridia are provided together in the form of a kit.

66. A composition comprising an effective dose of an antibiotic and an effective dose of at least one probiotic microorganism of the class clostridia which is resistant to said antibiotic drug.

67. The method of claim 48, wherein said antibiotic drug and said probiotic microorganism of the class clostridia are provided together in the form of a composition.
